(19) United States
(12) Patent Application Publication
(10) Pub. No.: US 2006/0195164 A1
(43) Pub. Date: Aug. 31, 2006

(54) Electro-therapeutic device and method of electro-therapeutic treatment

(76) Inventors: Jorgen Christian Sondergaard, Taastrup (DK); Birthe Sondergaard, Taastrup (DK)

Correspondence Address: GIFFORD, KRASS, GROH, SPRINKLE & CITKOWSKI, P.C, PO BOX 7021, TROY, MI, 48007-7021, US

(21) Appl. No.: 10/541,105

(22) PCT Filed: Dec. 18, 2003
(86) PCT No.: PCT/DK03/00891

§ 371 Date: Apr. 28, 2006

(30) Foreign Application Priority Data
Jan. 8, 2003 (DK) PA 2003 00009

(51) Int. Cl. A61N 1/18; A61N 1/34

(52) U.S. Cl. 607/76; 607/46

(57) ABSTRACT

There is provided an electro-therapeutic device having first and second electrodes or probes for making electrical contact to the body of an individual. The device has voltage supplying means for supplying an alternating output voltage across the electrodes to pass an alternating current through the body of the individual, and the voltage supply means are adapted for controlling the frequency of the output voltage so that the output voltage frequency is automatically changing in time between a low frequency and a high frequency. The voltage supply means may be adapted for controlling the frequency of the output voltage so that the output voltage frequency is changing between a low frequency and a high frequency at regular time intervals. There is further provided a method of applying an electrical stimulation signal to the body of an individual, where the method comprises providing an electrical stimulation signal with an electrical current having an AC component, where the AC component is changing in time between a low frequency and a high frequency with said high frequency being higher than said low frequency. The electrical stimulation signal is applied to a selected point of contact on the body of the individual in a manner to pass the electrical current through the selected point of contact on the body.

### FIELD OF THE INVENTION

The present invention relates to an electro-therapeutic device and a method of electro-therapeutic treatment wherein a small electric current is applied to the body.

### BACKGROUND OF THE INVENTION

The application of small currents to particular points of the body, such as acupuncture points, biologically active trigger points, neural junctions etc, has been found to provide relief for certain ailments. Such points may be characterised by having a low electrical resistance compared to adjacent areas of the body.

Electro-therapeutic devices, which can be held in the hand of a user, and which comprise a first active electrode for applying an electrical pulse to a selected portion of the skin of the user, are described in GB 1,416,141, U.S. Pat. No. 4,180,079 and U.S. Pat. No. 5,251,637. From these patents it is also known to have a second electrode arranged in a casing holding the device, in which second electrode current may flow in a return path through the hand that is holding the device. From GB 1,416,141 and U.S. Pat. No. 5,251,637 it is known to have a device comprising circuitry for measuring changes in the electrical resistance of the body when the device is moved across the skin of the body, and it is known to have an indication of a low resistance point of contact. From GB 1,416,141 it is known to have a visible indication of the low resistance point, and from U.S. Pat. No. 5,251,637 it is known an audible indication, where the audible signal changes volume or pitch as a function of the resistance.

From U.S. Pat. No. 4,180,079 it is further known to have an electrical signal, which is composed of two output signals having different frequencies. Here, the first signal is a low frequency signal of about 0.25 Hz and the second signal is a high frequency signal in the range 0.6-320 Hz. The low frequency signal has greater amplitude than the high frequency signal, and the two frequency signals are oscillating at the same time with the output signal being the difference of these two signals. However, since the two frequency signals are oscillating at the same time, it is not know to a have an output signal, which for a part time is oscillating at a low frequency and for another part time is oscillating at a higher frequency.

An apparatus, known as HealtTouch®, has been on the market for some years. This apparatus is capable of delivering an output signal of 2 Hz, 10 Hz or 100 Hz. However, the switching from one frequency to another is done manually by the user. Thus, it is not possible from this apparatus to achieve an output signal, which automatically changes in frequency during use.

It has been found by the present inventor that improved therapeutic results may be obtained when the electrical stimulation signal is changing in time between a low frequency and a high frequency. Thus, there is a need for an electro-therapeutic device, which can provide such a frequency changing electrical stimulation signal.

### SUMMARY OF THE INVENTION

According to the present invention, there is provided an electro-therapeutic device comprising:

first and second electrodes or probes for making electrical contact to the body of an individual,

voltage supplying means for supplying an alternating output voltage across said electrodes to pass an alternating current through the body of the individual, said voltage supply means being adapted for controlling the frequency of the output voltage so that the output voltage frequency is automatically changing in time between a low frequency and a high frequency, said high frequency being higher than said low frequency.

It is preferred that the voltage supply means is adapted for controlling the frequency of the output voltage so that the output voltage frequency is changing between a low frequency and a high frequency at regular time intervals. It is also preferred that the voltage supply means is adapted for controlling the frequency of the output voltage so that the output voltage is changing in time between one or more time periods having a low frequency and one or more time periods having a high frequency.

Different frequency ranges may be used, but preferably the low output voltage frequency should be in the range of 0.5-10 Hz, or in the range of 1-5 Hz. Here, the low output voltage frequency may be about 2 Hz or about 3 Hz. According to an embodiment of the invention, the high output voltage frequency may be in the range of 12-50 Hz or in the range of 15-40 Hz. Here, the high output voltage frequency may preferably be about 15 Hz or about 20 Hz. According to another embodiment of the invention the high output voltage frequency may be in the range of 40-300 Hz, in the range of 60-200 Hz, or in the range of 75-150 Hz. Here, the high output voltage frequency may preferably be about 100 Hz.

It is preferred that the voltage supply means is adapted for controlling the frequency of the output voltage so that the frequency of the output voltage is changed in cycles, each cycle comprising a first time period of low frequency and a second time period of high frequency. Here, a cycle time defined by the total time of the first time period and the second time period may be in the range of 2-25 seconds, in the range of 3-15 seconds, in the range of 4-10 seconds, or in the range of 5-6 seconds. Here, the cycle time defined by the total time of the first time period and the second time period may preferably be about 6 seconds.

According to an embodiment of the invention the low frequency may be supplied in a time period of low frequency being in the range of 1-6 seconds or in the range of 2-4 seconds. Here, the time period of low frequency may preferably be about 3 seconds.

Similarly, the high frequency may be supplied in a time period of high frequency being in the range of 1-6 seconds or in the range of 2-4 seconds. Here, the time period of high frequency may preferably be about 3 seconds.

It is preferred that the device of the present invention further comprises timing means for controlling the alternating output voltage to be applied for a predetermined time period.

The first electrode of the device of the invention may be an active electrode for making electrical contact to a selected point of the body of a patient, and the second electrode may be a passive electrode for making electrical contact over a relatively large area of the body of the individual when compared to the selected point area.

According to a preferred embodiment, the device of the invention comprises a casing which is holdable in the hand of an individual, said first electrode being mounted to the casing and said second electrode being disposed on the casing for making electrical contact with the hand of the individual. Here, the casing may be elongate and the first electrode may be mounted at one end of the casing, being electrically isolated from the body of the casing. It is preferred that at least part of the body of said casing is made of an electrically conducting material and the second electrode is in electrically contact with said electrically conducting part of the body of the casing.

The device of the invention may further comprise resistance detecting means for detecting when the first electrode is located at or near a low resistance point on the body of the individual, said resistance detecting means having means for detecting variations in the resistance between the first and second electrodes. Here, the device may further comprise means for providing an audible signal representative of the resistance. The means for providing an audible signal may be adapted to emit a sound, which changes in volume or pitch, the volume or pitch being proportional to or a function of the resistance. It is also within an embodiment of the invention that the device may comprise means for providing a visible signal representative of the resistance.

According to an embodiment of the invention the device may also comprise means for shifting between a standby mode and an active mode, wherein when in standby mode no alternating output voltage signal is supplied across the first and second electrodes and when in active mode, the alternating output voltage signal is supplied across the first and second electrodes. Here, the means for shifting between the standby mode and the active mode may be adapted to control said shifting as a function of current flowing between the first electrode and the second electrode. Thus, the mode shifting means may be adapted to hold the device in the standby mode when no current is flowing between the first and second electrodes. The mode shifting means may also be adapted to hold the device in the active mode when a current larger than or equal to a trigger current is flowing between the first and second electrodes. Here, the mode shifting means may comprise a power converter and resistor means, and the trigger current may generate a voltage drop across said resistor means whereby the power converter may shift from the standby mode to the active mode.

It is preferred that the voltage supplying means is adapted to supply an alternating output voltage. According to an embodiment of the invention the voltage may be in the range of 2-10 Volt, in the range of 3-8 Volt or in the range 4-6 Volt. Here, the voltage may preferably be about 5 Volt. According to another embodiment of the invention the voltage may be in the range of 10-50 Volt, in the range of 12-40 Volt, or in the range of 15-35 Volt. Here, the high output voltage frequency may preferably be about 20 Volt or 25 Volt.

It is also preferred that the voltage supplying means is adapted to pass an alternating current through the body of said individual in the range of 0.01-3 mA, or in the range of 0.02-1 mA.

In order to obtain a god electrical contact between the electrodes and the body of the user, it is preferred that the first and/or second electrodes have a conductive surface comprising a non-oxidising metal. Here, the non-oxidising metal may be selected from a group of materials comprising gold, silver and a platinum/chrome coating.

According to the present invention, there is also provided a method of applying an electrical stimulation signal to the body of an individual, said method comprising:

providing an electrical stimulation signal comprising an electrical current having an AC component, said AC component being changing in time between a low frequency and a high frequency with said high frequency being higher than said low frequency,

applying said electrical stimulation signal to a selected point of contact on the body of the individual in a manner to pass said electrical current through said selected point of contact on the body.

According to the present invention, there is furthermore provided a method of using an electrical stimulation signal for the alleviation of pain of an individual, said method comprising:

providing an electrical stimulation signal in the form of an electrical current having an AC component, said AC component being changing in time between a low frequency and a high frequency with said high frequency being higher than said low frequency,

applying said electrical stimulation signal to a selected point of contact on the body of the individual in a manner to pass said electrical current through said selected point of contact on the body, to thereby provide alleviation from said pain for said individual.

It is preferred that the electrical stimulation signal is applied to the selected point of contact on the body of the individual in a manner to pass said electrical current through a part of the body from said selected point of contact to a reference point or area of the body. The electrical stimulation signal may be applied to several selected points of contact on the body of the individual. However, it is preferred that the electrical stimulation signal is applied to one selected point at a time.

According to embodiments of the methods of the invention, the AC component may be changing between a low frequency and a high frequency at regular time intervals. It is also within the methods of the invention that the AC component is changing in time between one or more time periods having a low frequency and one or more time periods having a high frequency.

The low frequency of the AC component may be in the range of 0.5-10 Hz or in the range of 1-5 Hz. Preferably, the low frequency may be about 2 Hz or 3 Hz. According to one embodiment, the high frequency of the AC component may be in the range of 12-50 Hz or in the range of 15-40 Hz. Here, the high frequency may preferably be about 15 Hz. According to another embodiment, the high frequency of the AC component may be in the range of 40-300 Hz, in the range of 60-200 Hz, or in the range of 75-150 Hz. Here, the high frequency may preferably be about 100 Hz.

According to embodiments of the methods of the invention, the frequency of the AC component may be changed in cycles, each cycle comprising a first time period of low frequency and a second time period of high frequency. Here, a cycle time defined by the total time of the first time period and the second time period may be in the range of 3-15 seconds, in the range of 4-10 seconds, or in the range of 5-6 seconds. In a preferred embodiment the cycle time defined by the total time of the first time period and the second time period may be about 6 seconds.

According to the methods of the invention the low frequency may be supplied in a time period of low frequency being in the range of 1-6 seconds or in the range of 2-4 seconds. It is preferred that the time period of low frequency is about 3 seconds. Similarly, the high frequency may be supplied in a time period of high frequency being in the range of 1-6 seconds or in the range of 2-4 seconds. Preferably, the time period of high frequency may be about 3 seconds.

According to embodiments of the methods of the invention, the electrical stimulation signal may be applied to a selected point of contact for a predetermined time period. The electrical stimulation signal may be applied to one or more selected points of contact representing one or more low resistance points of contact on the body of the individual.

The methods of the invention may further comprise locating one or more selected points of contact representing one or more low resistance points of contact on the body of the individual.

It is preferred that the electrical stimulation signal is applied to the point(s) of contact via electrodes or probes, and the electrical stimulation signal may be applied to the point(s) of contact via first and second electrodes or probes. Here, the first electrode may be an active electrode for making electrical contact to a selected point of the body, and the second electrode may be a passive electrode for making electrical contact over a relatively large area of the body of the individual when compared to the selected point area.

It should be understood that the methods of the invention may include the use of an electro-therapeutic device selected from the embodiments of the present invention.

### BRIEF DESCRIPTION OF THE DRAWINGS

The invention will be further described in the following with the aid of the accompanying drawing, in which:

FIG. 1 is a schematic view of an embodiment of an electro-therapeutic device according to the invention,

FIG. 2 is a block diagram illustrating the functions of the circuitry of an embodiment of an electro-therapeutic device according to the invention,

FIG. 3 is a detailed circuit diagram corresponding to the block diagram of FIG. 2,

FIG. 4 is a flow chart illustrating the operation in time of an embodiment of an electro-therapeutic device according to the invention, and

FIG. 5 is a flow chart illustrating the program steps performed by a micro-controller being part of the circuitry of an embodiment of an electro-therapeutic device according to the invention.

### DETAILED DESCRIPTION OF PREFERRED EMBODIMENT

One of the principles of operation of an electro-therapeutic device in accordance with the present invention is that an improved therapeutic effect may be obtained when switching between a low frequency and a higher frequency in the stimulation signal, where the stimulation signal may be applied to so-called acupuncture skin points.

A further principle of operation of an electro-therapeutic device in accordance with the invention is that acupuncture skin points generally has a lower resistivity relative to skin points, which are not acupuncture points. This information can be transformed into an audible message, which gives the therapist a feedback signal facilitating the localisation of the acupuncture points. At the same time, the electrical excitation of the tip relative to the reference electrode applies stimulation to the located or selected acupuncture skin point. In addition to the audible feedback, there may also be a sensory feedback due to a therapeutic effect experienced by the user or patient, further optimising the therapy efficiency.

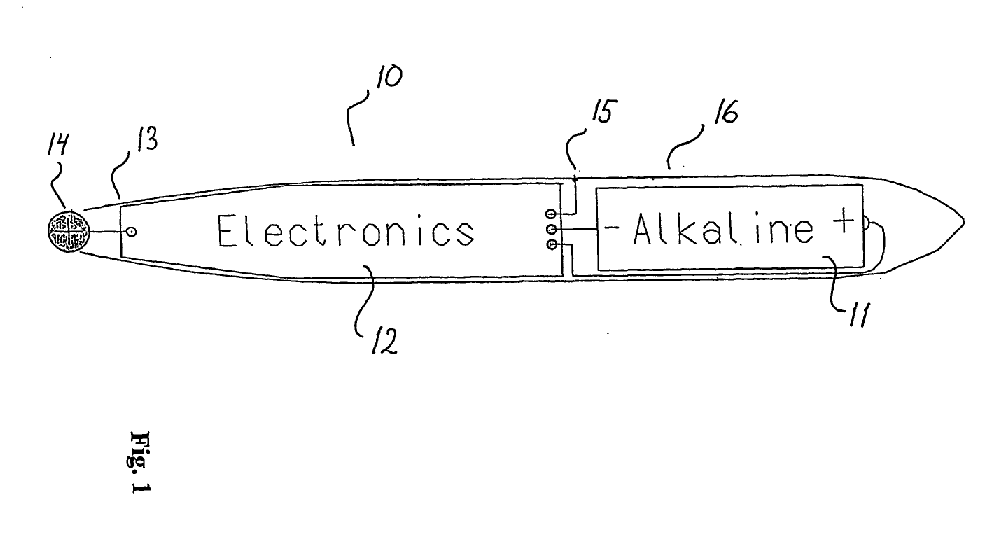

Referring to FIG. 1, an electro-therapeutic device in accordance with the invention comprises a casing 10, which is made by die-casting aluminium/magnesium with chrome/silver coating. The casing 10 houses a battery 11 and electronic circuitry 12 for supplying an alternating output voltage signal, which can be supplied to the body of an individual. The casing 10 is elongate and ends at one end in a nose 13, which carries a first, active electrode 14, the stimulation-tip. It is preferred that the stimulation-tip or electrode 14 is made with a rounded end having a radius of curvature comparable to the size of an acupuncture point on the skin of a body, and that the electrode 14 has a conductive surface made of a non-oxidising metal such as gold or silver. According to a preferred embodiment, the stimulation-tip may have a diameter of about 3 mm.

The stimulation-tip 14 is electrically isolated from the casing 10, and is connected to a first output of the electronic circuitry 12, which circuitry 12 has a second output connected to a second, passive electrode 15, being formed of an outer part of a lower end 16 of the casing 10. The second electrode 15 thereby may present a large, exposed contact surface area, which is positioned at a location where the fingers of a user may be positioned when the electro-therapeutic device of the invention is in use, thereby making a good electrical contact to the body of a user. The part of the lower end 16 forming the second electrode 15 may have a conductive surface of a non-oxidising metal such as gold, silver or a platinum/chrome coating.

The first electrode 14 serves as an active contact to a selected point of the body of the user with a stimulation current or signal being applied via the electrode 14, and the second electrode 15 forms the reference contact to the body relative to which the stimulation signal is applied. It is also possible to attach a wire to the second electrode 15, which wire on the other end, connects to the body of the patient through a good electrical contact such as a handle with a conductive surface of a non-oxidising metal as previously described. This is in particular useful, when the stimulation therapy is not performed by the patient himself, but by another person, thus ensuring that there is always an electrically good reference connection.

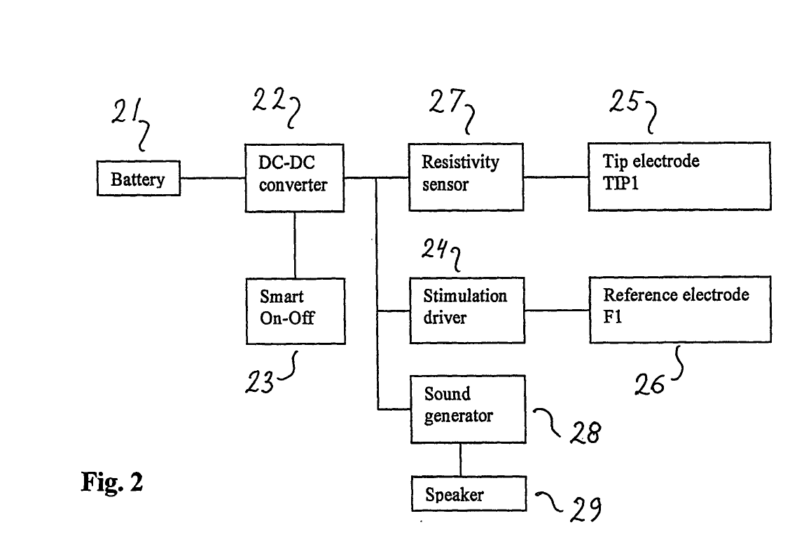

A block diagram illustrating the functional units of the circuitry of an embodiment of an electro-therapeutic device 20 according to the invention is shown in FIG. 2. The device 20 according to FIG. 2 comprises the following units: a battery 21, a DC-DC converter 22, a smart or intelligent on-off switch function 23, a stimulation driver function 24, a stimulation-tip electrode 25, which corresponds to the first electrode 14 as illustrated in FIG. 1, a reference contact electrode 26, which corresponds to the second electrode 15 as illustrated in FIG. 1, a resistivity sensor function 27, a sound generator function 28, and a loud-speaker 29. The block diagram illustrates the relation between the units.

The battery 21 is a single battery of type AAA with a nominal voltage of 1.5V and suitable capacity. The DC-DC converter 22 converts the battery voltage to a voltage suitable for feeding the electronic circuitry and to a voltage, which may optimise the acupuncture point stimulation effect. These two voltages are not necessarily the same, and in this preferred embodiment they are not equal. The on-off switch function 23 switches between normal supply to the electronics and a state with no power drain from the battery. After 5 sec. of normal power supply, the on-off switch function 23 automatically switches off the loud-speaker 29, when there is no skin-contact to the stimulation-tip electrode 25. This helps conserve power and minimise unnecessary noise. Normal operation with normal power supply is achieved when skin-contact is again obtained for the stimulation-tip electrode 25. The stimulation driver 24 modulates the voltage on the reference contact electrode 26 relative to the voltage on the stimulation-tip electrode 25, such that it induces a varying current in a selected skin point of the body of a user, which selected point may be an acupuncture point. It is preferred that the frequency of modulation is set to one or more frequencies, which may optimise the acupuncture point stimulation effect. The stimulation-tip electrode 25, 14 and the reference contact electrode 26, 15 have been discussed in connection with FIG. 1.

The resistivity sensor function 27 comprises a circuitry, which measures a stimulation current drawn by the stimulation-tip electrode 25 and relates it to an applied stimulation voltage, resulting in a measure of the resistivity of the selected skin point, which is an indication of the proximity of the skin point to an acupuncture point. When the tip electrode 25 is located at an acupuncture point, the electrical resistance between the reference electrode 26 and the tip electrode 25 is low compared to the situation, when the tip is located at a skin point, which is not an acupuncture point. The resistivity sensor function 27 is adapted to measure the electrical resistance between the tip electrode 25 and the reference electrode 26. The sound generator function 28 produces an excitation signal for the loud-speaker 29. It is preferred that the frequency is varied as a function of the resistivity being output from the resistivity sensor function 27. The amplitude of the excitation signal may preferably be fixed, but the amplitude could also be made user selectable. The loud-speaker 29 converts the excitation signal from the sound generator 28 into an audible message to the user or therapist.

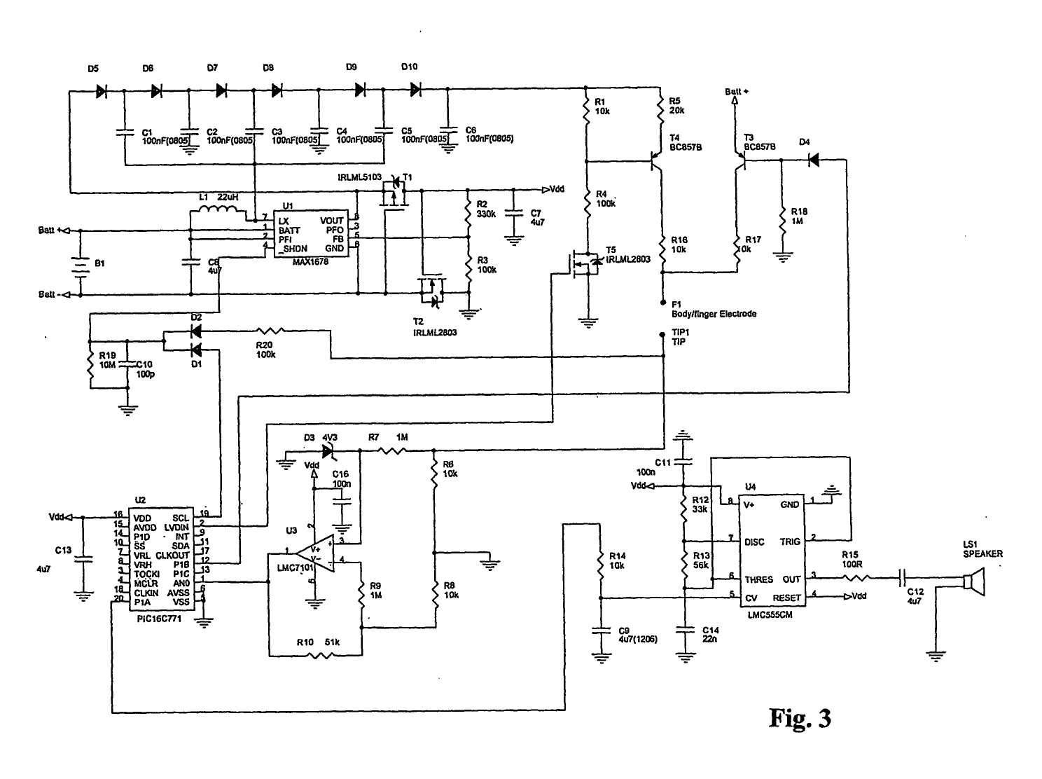

An embodiment of a detailed circuit diagram corresponding to the block diagram of FIG. 2 is shown in FIG. 3, a discussion of which will be given in the following.

The electro-therapeutic device of the invention may have two modes of operation: active and standby. Here, active, is the mode used during search-and-stimulation, while in standby mode the power consumption is minimized, but the circuit is nevertheless monitoring the stimulation-tip electrode 25 and the reference electrode 26 with the purpose of detecting a users intention of entering into an active search-and-stimulation mode.

To support these modes of operation the circuit in the diagram of FIG. 3 may be used. Here, the functioning of MOSFET's T1, T2 and transistor T3 but also the function of diodes D1 and D2 are central in the switching between the two modes of operation.

Power Conversion

The source of energy, which can be of any electric type delivering a voltage in the range of 0.9 to 5.5Volt from a low impedance source such as a electro chemical battery, rectifier system, solar cells or similar, enters the circuit though the terminals labelled Batt+ and Batt−. This battery voltage, which may be varying slightly due to for instance wear, is here converted to a fixed voltage independent of the source voltage by means of a commercially available switch-mode converter U1 (MAX1678) under the support of an energy storing inductor L1. The effective impedance of the power source is lowered by a capacitor C8. The generated stable voltage is available at the VOUT and GND pins of U1 when the pin4 of U1 labelled_SHDN is above 80% of the battery voltage relative to pin 6 GND of U1. The voltage value can be selected by the values of resistors R2 and R3, and in this particular configuration with R2 equal to 330 kΩ and R3 equal to 100 kΩ, the resulting voltage is 5V. This is the voltage value in the active mode. When pin 4 of U1 (_SHDN) is biased at a voltage lower than 20% of the battery voltage relative to GND of U1, the output voltage pin 8 of U1 is switched off, attaining a voltage of only about 1V. This is the voltage value under standby mode.

Stimulation Voltage Generator

The stimulation voltage, applies to the finger electrode, F1, through the resistor R5, transistor T4 and resistor R16. This stimulation voltage, at around 20 Volt, is generated from a circuit formed by the diodes D5, D6, D7, D8, D9 and D10, and from the capacitors C1, C2, C3, C4, C5 and C6. The inductor L1 connected to pin 7, LX, of the switch-mode converter U1 (MAX1678) generates a pulse that on the rising edge, with C1, C3 and C5 will charge the capacitors C2, C4 and C6 through the respective diodes D6, D8 and D10.

On the negative edge of the pulse, the capacitors C1 is charged to around 5 volt, that is the output voltage from pin 8 of U1. On the rising edge of the pulse, the voltage, in respect to GND, is rising to the sum of the voltage above the capacitor, around 5 volt, and the voltage above the inductor L1, around 5 volt, giving around 10 Volt at the anode of D6. The Diodes are connected in such a way that the current only can flow in one direction, and thereby increasing the voltage to around 10 volt at diode D6, around 15 volt at diode D8 and around 20 volt at diode D10. The result, when drawing a small current compared to the impedance of the capacitors, is around 20 Volt above C6 that is the source for the stimulation voltage. As the peak voltage over L1 can be slightly higher than 5 volt, it is compensating for the voltage drop over the diodes, giving a final value close to 20 Volt.

Sound Generator

A commercially available circuit, LMC555CM, here labelled U4 is coupled, together with resistors R12, R13 and a capacitor C14 as an astable multivibrator, also simply called an oscillator, but with the added effect that the frequency of operation depends on the voltage applied to the CV input of U4. This mode of operation is directly defined in the data-sheet supplied by the vendor of the LMC555CM circuit U4. The overall functionality may also be nominated a voltage controlled oscillator. The frequency range may be adapted to the human ears for maximum sensitivity. A square-wave signal on pin 3 of U3 labelled OUT is applied to a loudspeaker HT1 via a high-pass filter consisting of a capacitor C12. The high-pass is essential to avoid DC current in the loudspeaker coil, but could be eliminated when using for instance piezoelectric loudspeakers. A resistor R15 is limiting the current, and must be adapted to the type of loudspeaker being used. The voltage controlling the frequency (the voltage on pin 5 labelled CV on U4) is coming from a micro-controller U2 via a low-pass filter consisting of a resistor R14 and a capacitor C9. The purpose is to attenuate rapid variations emanating of this particular type of digital-to analogue converter (a Pulse Width Modulator), which is present in the micro-controller U2, which is here chosen as a commercially available controller PIC16C771.

Stimulation and Conductivity Measurement Circuit

Stimulation in form of varying voltage level that is applied to the finger electrode, F1, (here F1 corresponds to the reference electrode 26 in FIG. 2), via T4 that are conducting when its base electrode is pulled low by T5 trough resistor R4, that is controlled by pin 2 of U2 pin labelled LVDIN. This voltage is in a circuit formed by the tip electrode, TIP1, skin at tip, body, skin at finger-electrode and finger or body electrode, F1 (here F1 corresponds to the reference electrode 26 in FIG. 2), and giving rise to a current depending on the skin and body conductivity. The current flow results in a voltage drop across a resistor R6, which voltage is proportional to the current. In case the resistance is very low, the resistor R5 will limit the current by lowering the voltage at the emitter of T4, relative to the base. An operational amplifier U3, here LMC7101BIM5, in conjunction with resistors R10, R9, R7 and R8, forms a non-inverting amplifier effectively measuring the voltage across resistor R6 and supplying a voltage at pin 1 of U3, which may be around 5 times the voltage across resistor R6. The diode D3 may be needed in order to ensure that the voltage into U3 pin 3 is limited, even in the presence of an electrochemical induced voltage in the skin or by electrostatic discharge to electrode contacts.

Active Mode

In active mode, MOSFETS T1, and T2 are conducting, due to an applied gate-source voltage. In practice this means that they act as resistors in the drain-source channel, but attaining a value so small that in this application they effectively act as a short circuit of drain-source. The result is that the 5V supply and GND is connected to the rest of the circuitry. The tip electrode, TIP1, is connected to GND through the resistor R6, whereas the stimulation voltage can be flowing through T4 and R16 to the finger electrode F1.

Standby Mode

In standby mode, the MOSFETS T1 and T2 both enter the non-conducting state, effectively isolating the power converter and battery part from the rest of the circuitry, and in particular leaving the tip electrode TIP1 in a state where it is not influenced by the stimulation and conductivity measurement circuit.

Transition from Standby to Active Mode

When in standby mode a user touches both the finger electrode F1 and the tip electrode TIP1, a current is flowing from the battery positive terminal through transistor T3, through resistor R17, through the finger electrode F1, through the skin and body and into the tip electrode TIP1, through resistor R20, through diode D2 and through resistor R19 to the negative terminal of the battery. This current gives rise to a voltage across resistor R19, and charging the capacitor C10, which voltage is applied to the pin 4 (_SHDN) on U1. When this voltage is more than 80% of the battery voltage, the power converter U1 goes from shut-down to active mode, and starts generating the 5V supply voltage at its output, which in turn puts MOSFETS T1 and T2 in conduction mode, with the result that the micro-controller U2 starts executing its program, and as a first task applies a logic high level (about 4.5V) to pin 19 labelled SCL, which is connected to the diode D1. Diode D1 is conducting and will thus ensure that the voltage at pin 4 of U1 (_SHDN) will be above 80% of the battery voltage irrespective of the voltage at the tip electrode TIP1, thereby ensuring that the power converter remains in active mode. The capacitor C10 is ensuring that the voltage supplied to pin 4 of U1 (_SHDN) is maintaining this voltage during the transition from when the 5 volt supply is rising, and thereby disconnecting the voltage to the finger electrode F1, and until pin 19 U2 is high.

Transition from Active to Standby Mode

When the micro-controller U2 program wants to put the circuit in standby mode, it applies a low logic voltage at pin 19 and at the tip electrode TIP1 through pin 2 of U2, through transistor T5 and thereby bringing the transistor T4 into non-conducting state. This effectively brings the voltage at pin 4 of U1 (_SHDN) below 20% of the battery voltage, and thus brings the converter U1 to the shut-down mode. In shut-down mode, the voltage at U1's output, pin 8 (VOUT), is only one diode forward voltage drop below the battery voltage (this is an inherent characteristic of a boost switch mode DC-DC converter). I.e. with a single cell battery of 1.5V, U1's output voltage on pin 8 in shut-down is roughly about 1.0V. This in turn switches the MOSFET's T1 and T2 in non-conducting mode, removing power to the rest of the circuit, including the micro-controller U2. In effect, this mode is the standby mode, having a power consumption of only a few microwatts, but still the ability to monitor and react to the user's intention to start a search-and-stimulate session.

Micro-Controller U2, PIC16C771

The central component of the circuitry of FIG. 3 is the micro-controller U2 (which here is the PIC16C771 from Microchip). U2 is programmed to perform the resistivity calculation, to control and apply the stimulation voltage, to generate the signal for the loudspeaker and to interface with the user for setting of a suitable stimulation frequency. It contains a non-volatile ram, which is used to maintain the preferred settings from one séance to another. It contains a timer circuitry, which serves as a reference clock for generation of the stimulation frequency and for the audible signal as well as for the on-off functionality, switching between active and standby. It contains an analogue-to-digital converter, which converts the current measurements into a digital representation suitable for numeric calculations. It also contains the necessary non-volatile program memory, which in addition can be reprogrammed in a suitable programming station, even after the components has been mounted on the circuit board, opening the possibility of firmware upgrading or last-minute production modification with very low additional costs. The potential of offering different versions distinct by for instance audible characteristics, is a possibility, which could be considered.

When the tip electrode TIP1 is located at an acupuncture point, the electrical resistance between finger electrode F1 and tip electrode TIP1 is low compared to the case where the tip electrode TIP1 is located at a point which is not an acupuncture point. The electrical resistance between the finger electrode F1 and the tip electrode TIP1 is measured. This is done indirectly by first applying a voltage drop between the finger electrode F1, the tip electrode TIP1, and a configuration of resistors, in such a way that measuring a voltage drop across the resistor R6 gives information on the resistance between the finger electrode F1 and tip electrode TIP1 to the micro-controller U2. The micro-controller U2 determines the voltage drop by use of an analog-to-digital converter (ADC), which is built into the microprocessor. On the basis of the measured voltage drop, an audio signal is generated so that the user can hear whether or not the tip electrode TIP1 is located at an acupuncture point. The audio signal is generated from pin 20 of U2, where the duty cycles can be set with up to 12 bit's accuracy, and via the low-pass filter R14, C9 into pin 5 of U4. In this construction, the circuit U4 is working as a frequency to voltage converter, together with the low-pass filter, R14, C9.

The electro-therapeutic device of the present invention is not adapted to find acupuncture points only, but the micro-controller U2 of the device is programmed so as to control a stimulation of the acupuncture points by an oscillating voltage drop between the finger electrode F1 and the tip electrode TIP1. The oscillating voltage may preferably shift between the oscillation frequencies 2 Hz and 100 Hz with intervals of 3 seconds. The output Voltage to the tip electrode TIP1 is here about 20Volt and the flowing current is about 0.01 mA-0.2 mA, depending on the actual resistance of the selected body point.

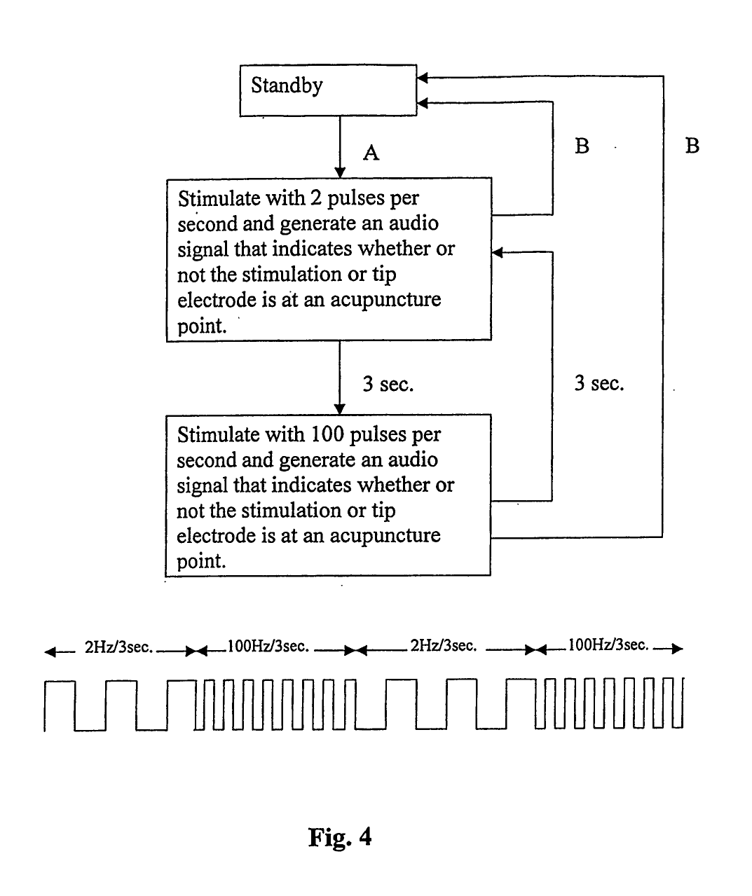

In FIG. 4 the operation in time of an embodiment of an electro-therapeutic device according to the invention is illustrated. Initially, the device is in the standby mode. When the conductivity between the electrodes F1 ands TIP1 (26 and 25) of the device is increased by contacting the tip electrode TIP1 to a body point, the device goes from standby to active mode as described above. This is indicated by an A in FIG. 4. When in the active mode, the device is set to stimulate with 2 pulses per second and generate an audio signal that indicates whether or not the stimulation or tip electrode is at an acupuncture point having a high conductivity. After applying a 2 Hz signal for about 3 sec. the stimulation process proceeds to stimulate with 100 pulses per second for another 3 sec. and generate an audio signal that indicates whether or not the stimulation or tip electrode is at an acupuncture point. As long as the conductivity between the electrodes F1 and TIP1 is large enough, the device will shift every 3 sec. between the 2 Hz and the 100 Hz stimulation frequencies. If the conductivity between the electrodes F1 and TIP1 has been too low for about 5 sec., the device will enter into the standby mode.

In FIG. 4 is also shown the periodically shifting of the stimulation frequency between the low 2 Hz and the higher 100 Hz.

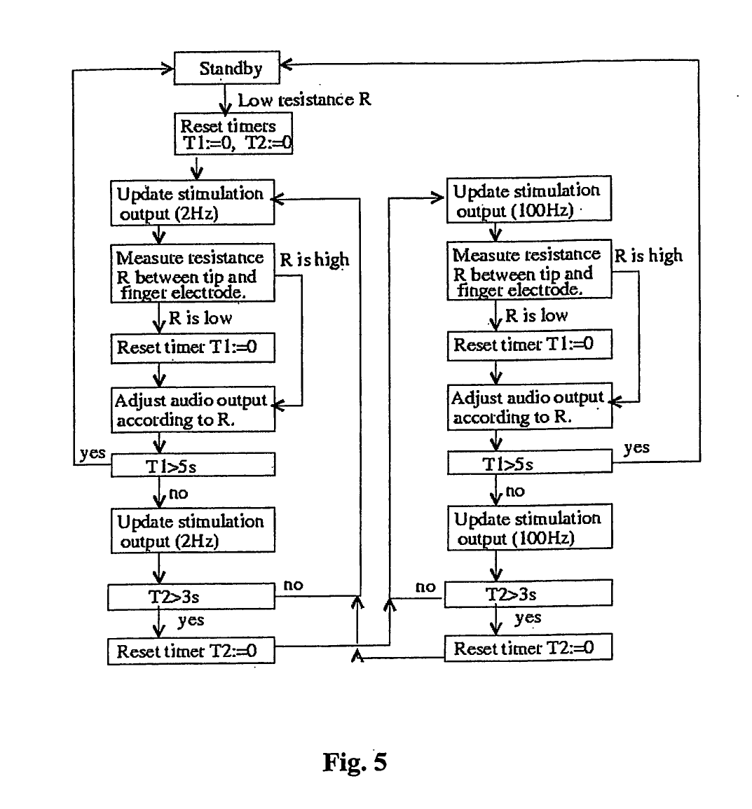

In FIG. 5 is shown a flow chart illustrating the program steps performed by the micro-controller being part of the circuitry of a preferred embodiment of an electro-therapeutic device according to the invention. The flow chart is described in the following:

Standby

The micro-controller starts in the state referred to in the flow-chart as “Standby”. In this state the electrical power to the electronics of the electro-therapeutic device is switched off. The device will remain in this state until an electrical connection is made between the finger or reference electrode (F1, 26) and the tip or stimulation electrode of the device (TIP1, 25). An electrical connection means in this case that the electrical resistance R between the finger or reference electrode (F1, 26) and the tip or stimulation electrode (TIP1, 25) becomes low. The resistance R becomes low when e.g. the user of the device holds on to the finger electrode F1 with his right hand and places the tip of the tip electrode (TIP1) somewhere else on his body. In both cases the tip and finger electrode (TIP1, F1) should be in contact with the skin of the user's body. As the resistance R becomes low, power will become available to the electronics, and in particular power will be available to the micro-controller. The micro-controller can control the power supply and make the power stay switched on even after the resistance R becomes high again.

Reset Counters or Timers T1:=0, T2:=0

The first operation of the micro-controller is to reset counters T1 and T2, which are used to keep track of times for switching between different operations.

Update Stimulation Output (2 Hz)

The micro-controller then updates the frequency of the stimulation voltage across the finger electrode (F1) and the tip electrode (TIP1) such that a stimulation signal is maintained with a stimulation frequency of 2 Hz.

Measure Resistance R between Tip Electrode (TIP1) and Finger Electrode (F1)

The micro-controller then obtains a measure of the resistance R between the finger electrode and the tip. This is done by use of measuring a voltage with an analogue-to-digital converter (ADC) built into the micro-controller as previously described. If the measured resistance R is sufficiently low, such that it is reasonable to assume that the device is in use, then the counter T1 is reset (T1:=0). If R is very large the counter T1 is not reset.

Adjust Audio Output According to Resistance R

According to the measure of R, the user is informed about the value of R via control of an audio output. The resistance R is much lower when the tip electrode (TIP1) is located at an acupuncture point as compared to when the tip electrode (TIP1) is located elsewhere on the skin of the user (we still assume that also the finger electrode (F1) is in contact with the skin of the user). It is therefore possible for the user of the device to hear whether or not the tip electrode (TIP1) is located at an acupuncture point.

T1>5 s

If the device has not been in use for more than 5 seconds (T1>5 s) the micro-controller will go back to the state “standby” where the electrical power is switched off. Although 5 seconds has been chosen here it is also possible to use e.g. 3 seconds, 10 seconds or any other choice of time limit. The important thing is that the device will switch off by itself within a reasonable time limit when it is not being used. The accuracy of the time limit of 5 s here may be on the order of 1 s. If the device has been in use (low R) within the most recent 5 seconds it will continue to the following state “Update stimulation output (2 Hz)”.

Update Stimulation Output (2 Hz)

The micro-controller again updates the frequency of the stimulation voltage across the finger electrode (F1) and the tip electrode (TIP1) such that a stimulation signal is maintained with a stimulation frequency of 2 Hz.

T2>3 s

If the counter T2 exceeds a period of three seconds (T2>3 s) the counter T2 will be reset and the operation of the device will continue with “Update stimulation output (100 Hz)”. Otherwise the operation will continue with “Update stimulation output (2 Hz)”. It is not very important that the stimulation with 2 Hz and 100 Hz are carried out for periods of exactly three seconds. It is important that the device changes between these stimulation frequencies for the stimulating signal such that the user will be stimulated with both frequencies within a reasonable time. A reasonable time is the time that a user can be expected to keep the tip electrode (TIP1) of the device located at a fixed acupuncture point or skin or body point.

The control loop steps for the 100 Hz mode is similar to the control steps for the 2 Hz mode:

Measure Resistance R between Tip Electrode (TIP1) and Finger Electrode (F1)

The micro-controller then obtains a measure of the resistance R between the finger electrode and the tip as already described. If the measured resistance R is sufficiently low, such that it is reasonable to assume that the device is in use, then the counter T1 is reset (T1:=0). If R is very large the counter T1 is not reset.

Adjust Audio Output According to Resistance R

According to the measure of R, the user is informed about the value of R via control of an audio output.

T1>5 s

If the device has not been in use for more than 5 seconds (T1>5 s) the micro-controller will go back to the state “standby” where the electrical power is switched off. Again it is also possible to use e.g. 3 seconds, 10 seconds or any other choice of time limit. If the device has been in use (low R) within the most recent 5 seconds it will continue to the following state “Update stimulation output (100 Hz)”.

Update Stimulation Output (100 Hz)

The micro-controller again updates the frequency of the stimulation voltage across the finger electrode (F1) and the tip electrode (TIP1) such that a stimulation signal is maintained with a stimulation frequency of 100 Hz.

T2>3 s

If the counter T2 exceeds a period of three seconds (T2>3 s) the counter T2 will be reset and the operation of the device will continue with “Update stimulation output (2 Hz)”. Otherwise the operation will continue with “Update stimulation output (100 Hz)”.

Various modifications may be made to the described embodiments, and it is desired to include all such modifications and mechanical and functional equivalents as fall within the scope of the accompanying claims.

1. An electro-therapeutic device comprising:
first and second electrodes for making electrical contact to the body of an individual,
a voltage supply for supplying an alternating output voltage across said electrodes to pass an alternating current through the body of the individual, said voltage supply being adapted for controlling the frequency of the output voltage so that the output voltage frequency is automatically changing in time between a low frequency and a high frequency, said high frequency being higher than said low frequency, and
a mode shifter for shifting said device between a standby mode and an active mode, wherein when in standby mode no alternating output voltage signal is supplied across the first and second electrodes and when in active mode, the alternating output voltage signal is supplied across the first and second electrodes, said mode shifter being adapted to control said shifting as a function of current flowing between the first electrode and the second electrode.

2. A device according to claim 1, wherein the mode shifter is adapted to hold the device in the standby mode when no current is flowing between the first and second electrodes.

3. A device according to claim 1, wherein the mode shifter is adapted to hold the device in the active mode when a current larger than or equal to a trigger current is flowing between the first and second electrodes.

4. A device according to claim 3, wherein the mode shifter comprises a power converter and resistor, and said trigger current generates a voltage drop across said resistor whereby the power converter shifts from a standby mode to an active mode.

5. A device according to claim 1, wherein the voltage supply is adapted for controlling the frequency of the output voltage so that the output voltage frequency is changing between a low frequency and a high frequency at regular time intervals.

6. A device according to claim 1, wherein the voltage supply is adapted for controlling the frequency of the output voltage so that the output voltage is changing in time between one or more time periods having a low frequency and one or more time periods having a high frequency.

7. A device according to claim 1, wherein the low output voltage frequency is in the range of 0.5-10 Hz.

8. A device according to claim 7, wherein the low output voltage frequency is in the range of 1-5 Hz.

9. A device according to claim 8, wherein the low output voltage frequency is about 2 Hz.

10. A device according to claim 1, wherein the high output voltage frequency is in the range of 12-50 Hz.

11. A device according to claim 10, wherein the high output voltage frequency is in the range of 15-40 Hz.

12. A device according to claim 11, wherein the high output voltage frequency is about 15 Hz.

13. A device according to claim 1, wherein the high output voltage frequency is in the range of 40-300 Hz.

14. A device according to claim 13, wherein the high output voltage frequency is in the range of 60-200 Hz.

15. A device according to claim 14, wherein the high output voltage frequency is in the range of 75-150 Hz.

16. A device according to claim 15, wherein the high output voltage frequency is about 100 Hz.

17. A device according to claim 1, wherein the voltage supply is adapted for controlling the frequency of the output voltage so that the frequency of the output voltage is changed in cycles, each cycle comprising a first time period of low frequency and a second time period of high frequency.

18. A device according to claim 17, wherein a cycle time defined by the total time of the first time period and the second time period is in the range of 3-15 seconds.

19. A device according to claim 17, wherein a cycle time defined by the total time of the first time period and the second time period is in the range of 4-10 seconds.

20. A device according to claim 17, wherein a cycle time defined by the total time of the first time period and the second time period is in the range of 5-6 seconds.

21. A device according to claim 17, wherein a cycle time defined by the total time of the first time period and the second time period is about 6 seconds.

22. A device according to claim 1, wherein a time period of low frequency is in the range of 1-6 seconds.

23. A device according to claim 1, wherein a time period of low frequency is in the range of 2-4 seconds.

24. A device according to claim 1, wherein a time period of low frequency is about 3 seconds.

25. A device according to claim 1, wherein a time period of high frequency is in the range of 1-6 seconds.

26. A device according to claim 1, wherein a time period of high frequency is in the range of 2-4 seconds.

27. A device according to claim 1, wherein a time period of high frequency is about 3 seconds.

28. A device according to claim 1, said device further comprising a timer for controlling the alternating output voltage to be applied for a predetermined time period.

29. A device according to claim 1, wherein the first electrode is an active electrode for making electrical contact to a selected point of the body of a patient, and the second electrode is a passive electrode for making electrical contact over a relatively large area of the body of the individual when compared to the selected point area.

30. A device according to claim 1, said device comprising a casing which is holdable in the hand of an individual, said first electrode being mounted to the casing and said second electrode being disposed on the casing for making electrical contact with the hand of the individual.

31. A device according to claim 30, wherein the casing is elongate and the first electrode is mounted at one end of the casing, being electrically isolated from the body of the casing.

32. A device according to claim 31, wherein at least part of the body of said casing is made of an electrically conducting material and the second electrode is in electrically contact with said electrically conducting part of the body of the casing.

33. A device according to claim 1, said device further comprising resistance detecting means for detecting when the first electrode is located at or near a low resistance point on the body of the individual, said resistance detecting means having means for detecting variations in the resistance between the first and second electrodes.

34. A device according to claim 33, said device further comprising means for providing an audible signal representative of the resistance.

35. A device according to claim 34, wherein the means for providing an audible signal is adapted to emit a sound which changes in volume or pitch, the volume or pitch being a function of the resistance.

36. A device according to claim 33, said device further comprising means for providing a visible signal representative of the resistance.

37. A device according to claim 1, wherein the voltage supply is adapted to supply an alternating output voltage having a voltage swing in the range of 2-10 V.

38. A device according to claim 1, wherein the voltage supply is adapted to supply an alternating output voltage having a voltage swing in the range of 10-50V.

39. A device according to claim 1, wherein the voltage supply is adapted to pass an alternating current through the body of said individual in the range of 0.01-3 mA.

40. A device according to claim 1, wherein the first and/or second electrodes have a conductive surface comprising a non-oxidising metal.

41. A device according to claim 40, wherein the non-oxidising metal is selected from a group of materials comprising gold, silver and a platinum/chrome coating.
